(19) United States
(12) Patent Application Publication
(10) Pub. No.: US 2023/0241324 A1
(43) Pub. Date: Aug. 3, 2023

(54) Point-of-Care Testing Apparatus

(71) Applicant: Drummond Scientific Company, Broomall, PA (US)

(72) Inventors: Edward F Farina, Lincoln University, PA (US); Nicholas DiTrolio, Havertown, PA (US); Christopher M Strohsahl, West Chester, PA (US); Scott Ferguson, Springfield, PA (US); Eric Smith, Springfield, PA (US); Peter Esslinger, Royersford, PA (US); Jeffrey DiTrolio, Newtown Square, PA (US); Jack A Walker, Kennett Square, PA (US)

(21) Appl. No.: 17/919,279

(22) PCT Filed: Apr. 16, 2021
(86) PCT No.: PCT/US2021/027813

§ 371 Date: Oct. 16, 2022

Related U.S. Application Data
(60) Provisional application No. 63/010,985, filed on Apr. 16, 2020.

(51) Int. Cl. A61M 5/315

(52) U.S. Cl. CPC A61M 5/31596; A61M 5/31533; A61M 5/31501; A61M 2005/3128

(57) ABSTRACT

An apparatus for collecting, diluting and dispensing a predetermined volume of a patient fluid sample, such as blood, into the collection port of a point-of-care diagnostic test. The apparatus is constructed and arranged to collect a predefined volume of fluid sample by wicking, to mix the whole blood with a dilution reagent, and to dispense a predefined volume (within predefined tolerances) of diluted fluid sample into the collection port of the POC test kit. The apparatus has an elongate body with an internal cavity and a capillary tube in fluid connection with the internal cavity. A mixing valve separates the capillary tube and the internal cavity and selectively opens and closes the fluid connection between the capillary tube and the internal cavity. A plunger reciprocates within the valve body between a retracted limit position and a distal limit position for creating negative and positive pressure within the internal cavity.

### FIELD OF THE INVENTION

The present invention relates to a point-of-care apparatus for use in performing a specialized diagnostic test, such as a test for the presence of pro-b-type natriuretic peptide. More particularly, the invention relates to an apparatus for collecting, diluting and dispensing a predetermined volume of a patient blood sample into the collection port of a point-of-care diagnostic test.

### BACKGROUND OF THE INVENTION

Traditional medical testing involves collection of a sample of a patient's blood, saliva, urine, etc. at or near the patient's time and place of care, i.e., “point of care.” The sample is then shipped to and tested at a remote medical laboratory, which requires the care giver to wait hours or days to learn the results. During that waiting period, patient care must continue without knowing the results of the diagnostic test. To address this problem, point-of-care (“POC”) medical test kits were developed and are now common. These POC test kits allow the care giver to perform many standard diagnostic tests at the patient's place of care. Most POC test kits are self-contained, fully-automated, and simply require the care giver to deposit the patient's fluid sample into a collection port on the kit. However, often the fluid sample must be diluted and must be a precise amount.

The Centers for Medicare & Medicaid Services (CMS) regulates all laboratory testing (except research) performed on humans in the U.S. through the Clinical Laboratory Improvement Amendments (CLIA). In total, CLIA covers approximately 260,000 laboratory entities. The Division of Clinical Laboratory Improvement & Quality, within the Quality, Safety & Oversight Group, wider the Center for Clinical Standards and Quality (CCSQ) has the responsibility for implementing the CLIA Program. The objective of the CLIA program is to ensure quality laboratory testing.

In order to obtain a CLIA waiver and get FDA approval for home-use of certain POC test kits, the kit must include a reliable fluid-sample collection apparatus so that the patient or caregiver can properly collect and deposit the patient fluid sample into the test-kit collection port. For example, for test requiring a patient blood sample, the collection apparatus must allow the point-of-care provider or patient to be able to perform the following tasks: (1) collect a predefined volume of whole blood from a fingerstick; (2) mix the whole blood with a dilution reagent; and (3) dispense a predefined volume (within pre-defined tolerances) of diluted blood into the sample collection port of the POC test kit. In order to obtain CLIA approval, the fluid-sample collection apparatus must be simple to use, and have insignificant risk of an erroneous result. The collection apparatus should work consistently independent of the collection technique of the caregiver, and independent of any sample manipulation. The collection apparatus should also be maintenance free and requires minimal or no operator intervention during sample collection or test analysis. When used for collecting blood samples, the collection apparatus should desirably be self-contained with pre-filled, pre-measured reagents. Because such POC test kits are mass produced and distributed in bulk, the collection apparatus should desirably have robust transport and storage capability in temperatures from about 2-40° C. Finally, to prevent cross-contamination, it is desirable that the collection apparatus can be used only once.

### SUMMARY OF THE INVENTION

The present invention relates to an apparatus for collecting, diluting and dispensing a predetermined volume of a patient blood sample into the collection port of a point-of-care diagnostic test. In one preferred embodiment, the apparatus is constructed and arranged to collect a predefined volume of whole blood from a fingerstick, mix the whole blood with a dilution reagent, and dispense a predefined volume (within pre-defined tolerances) of diluted blood into the sample collection port of the POC test kit within. The fluid-sample collection apparatus is simple to use, and has insignificant risk of an erroneous result. The fluid-sample collection apparatus works consistently independent of the collection technique of the caregiver, and independent of any sample manipulation. The collection apparatus is maintenance free and requires minimal operator action during sample collection and transfer to the POC diagnostic test kit. In a preferred embodiment, the collection apparatus is pre-filled with a precise amount of diluting reagent and can be used only once.

In one preferred embodiment, the apparatus generally comprises an elongate, cylindrical main body, having a longitudinally-extending central axis, a distal end, a proximal end, and an internal cavity. A capillary tube is fixed at the distal end of the main body. A mixing valve is located intermediate the capillary tube and the internal cavity for selectively connecting and disconnecting a fluid-flow channel between the capillary tube and the internal cavity. A plunger reciprocates within the main body between a retracted limit position and a distal limit position for creating negative and positive pressure within the internal cavity.

The apparatus may include a nosepiece on the distal face of the mixing valve. The nosepiece may be constructed and arranged to releasably connect the capillary tube in fluid connection with the mixing valve. In that embodiment, the nosepiece may be made of an elastomeric material and has a socket with an inner diameter slightly smaller than the outer diameter of the capillary tube so that a compressive force connects the capillary tube when the capillary tube is forced into the socket.

The mixing valve has an open position and a closed position, and a vent channel in fluid connection with the capillary tube when the mixing valve is in the closed position. The mixing valve enables admission of the blood sample into the capillary tube and isolates the capillary tube from the internal cavity in the closed position, and enables the blood sample to flow from the capillary tube into the internal cavity in the open position.

In one preferred embodiment, the mixing valve includes a valve body having distal, proximal and lateral side walls, a central channel, a distal and proximal bore extending through the distal and proximal wall, respectively, of the valve body that are aligned with said central axis. A valve gate having a size and shape that are complimentary to the central channel is movable within the central channel of the valve body. The valve gate has a fluid-flow bore extending therethrough that can align with the distal and proximal bores in the valve body in the open position.

An actuator button is fixed to one end of the valve gate. When depressed, the actuator button moves the valve gate from the closed positon to the open position. The mixing valve is in the open position when the actuator button is depressed to an open limit position abutting the top of the valve body. In this position, the actuator button is generally flush with the valve body, which makes it very difficult to pull back since there are no exposed edges to grasp. This construction thereby makes it very difficult to move the valve back to the closed position for re-using the apparatus. In another preferred embodiment, the valve gate engages a detent in the open position, which makes it even more difficult to move back to the closed position.

In one preferred embodiment, the plunger comprises an actuator stem, a seal seated at the distal end of the stem, and a handle fixed to the proximal end of the stem. The plunger also preferably includes an expulsion rod fixed to the distal end of the actuator stem. The rod has a diameter slightly smaller than the inner diameter of the capillary tube. Preferably, the expulsion rod has a chamfered distal nose and a flat surface that extends along the length of the expulsion rod. The expulsion rod is arranged co-axially with the central axis so that the expulsion rod extends through the fluid-flow channel in the mixing valve and the capillary tube when the plunger is moved to its distal limit position. The expulsion rod is preferably made from Teflon so that it will pass smoothly through the transitions between the main body, mixing valve, and capillary tube.

In another preferred embodiment, the apparatus comprises a main body having a longitudinally-extending central axis, a distal end, a proximal end, and an internal cavity containing a diluting solution. A capillary tube is connected at said distal end of said body. A valve is connected intermediate the capillary tube and the internal cavity for selectively connecting and disconnecting a fluid-flow connection between the capillary tube and the internal cavity.

The apparatus includes means for transferring the fluid sample in the capillary tube into the internal cavity, first means for mixing the fluid sample and diluting solution within the cavity to form a diluted fluid sample, and means for expelling the diluted fluid sample from the internal cavity and into the capillary tube. In preferred embodiments, mixing of the fluid sample and diluting solution is achieved by creating a vacuum in the internal cavity and then creating a fluid flow connection with the capillary tube. This pressure differential causes turbulent fluid flow from the capillary tube into the internal cavity and causes the fluids to mix.

The apparatus further includes means for expelling the diluted fluid sample from the capillary tube. In preferred embodiments, expulsion is achieved by pressurizing the internal cavity to force the diluted fluid sample through the valve and out the capillary.

In another preferred embodiment, the apparatus includes second means for mixing the diluted fluid sample within the capillary tube as it is being expelled. In preferred embodiments, secondary mixing is achieved by reducing the cross sectional area of the capillary tube to increase fluid flow turbulence. In one embodiment, an expulsion rod is inserted into the capillary tube, and the diluted fluid must flow through the gap between the outer surface of the expulsion rod and the inner surface of the capillary tube.

The apparatus also preferably includes means for venting the capillary tube as the diluted fluid sample is being expelled from said capillary tube. In the embodiment wherein an expulsion rod is inserted in the capillary tube, the rod has a flat surface along it length, which allows venting of the diluted fluid sample through the capillary tube.

The apparatus preferably includes means for preventing the apparatus from being re-used by preventing the valve from being closed once it is opened. In one preferred embodiment, a detent engages the valve gate in the open position, which makes it very difficult to move the valve back to the closed position. In another preferred embodiment, the valve can be moved easily from the closed position to the open position by depressing a first end of a slidable valve gate; however, the opposed second end of the gate cannot be depressed to move the valve gate back to the closed position because it does not extend outside the valve body, and the first end of the gate has a shape that makes it very difficult to grasp or pull back to the closed position.

### BRIEF DESCRIPTION OF THE DRAWINGS

FIG. 1 is a perspective view of an apparatus in accordance with a preferred embodiment of the invention;

FIG. 2 is an enlarged, perspective view of the distal end of the apparatus of FIG. 1;

FIG. 3 is an enlarged, perspective view of the mixing valve, nose, and capillary tube of the apparatus of FIG. 1;

FIGS. 4a and 4b are perspective views of the distal end of the apparatus of FIG. 1 showing the mixing valve in the closed and open positions, respectively;

FIGS. 5a, 5b and 5c are top views with the valve gate removed showing the plunger in the retracted position, an intermediate position, and the extended position, respectively;

FIG. 6 is a top plan view of the apparatus of FIG. 1;

FIG. 7 is a section taken along lines 7-7 of FIG. 6;

FIG. 8 is a perspective view of the distal end of the plunger of the apparatus of FIG. 1;

FIG. 9 is a perspective view of the valve gate of the apparatus of FIG. 1;

FIG. 10 is a perspective view of apparatus in accordance with another preferred embodiment of the invention;

FIG. 11 is an enlarged perspective view of the valve of FIG. 10;

FIG. 12 is a cross-sectional view of the apparatus of FIG. 10; and,

FIGS. 13a and 13b are perspective views of the valve of FIG. 10 in a closed position and an open position, respectively.

### DETAILED DESCRIPTION OF PREFERRED EMBODIMENTS

For the purpose of illustrating the invention, an embodiment of the invention is shown in the accompanying drawings. However, it should be understood by those of ordinary skill in the art that the invention is not limited to the precise arrangements and instrumentalities shown therein and described below. Throughout the specification, like reference numerals are used to designate like elements. Numerous changes and modifications within the spirit and scope of the invention will become apparent to those skilled in the art from this detailed description.

Unless otherwise defined, all technical and scientific terms used herein in their various grammatical forms have the same meaning as commonly understood by one of ordinary skill in the art to which this invention belongs. With reference to an elongate element or series of elements, the term “proximal” means the element or portion of element closest to the operator when the invention is use for its intended purpose, and the term “distal” means the element or portion of element farthest from the operator. In the embodiment shown herein, the distal end refers to the end from which the capillary tube extends and the proximal end refers to the end from which the plunger stem extends.

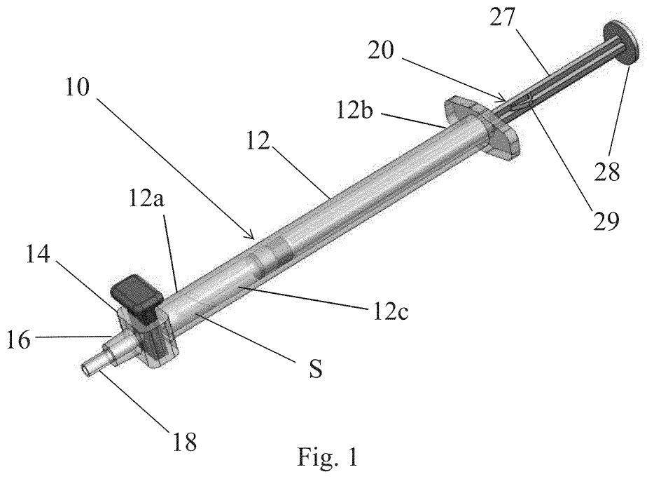

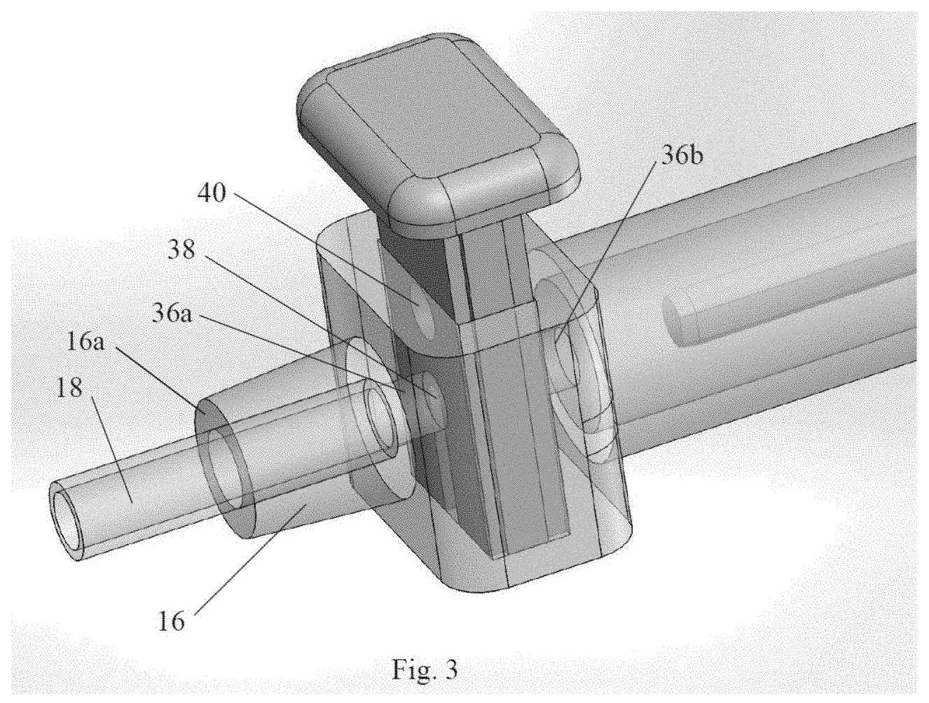

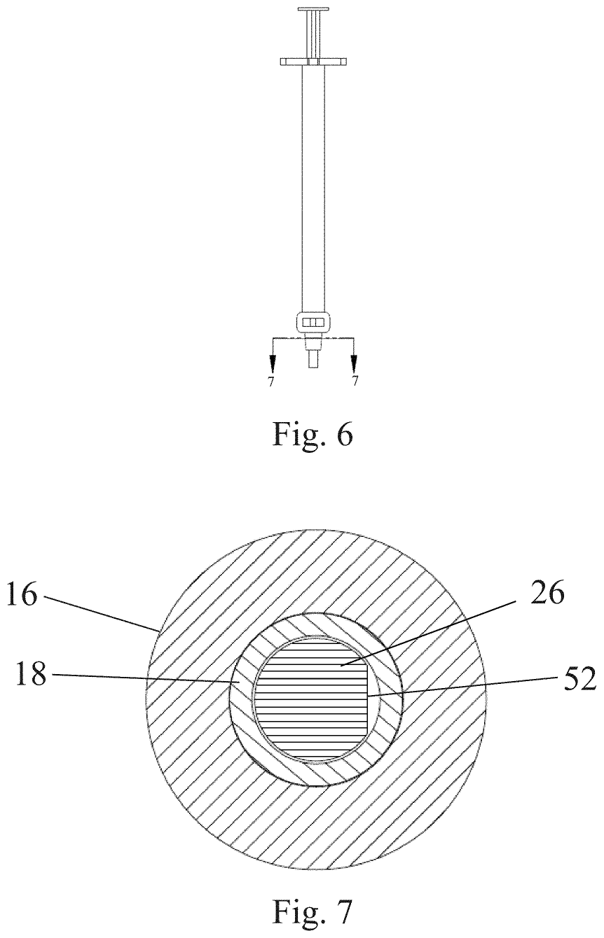

A fluid-sample collection apparatus for use in connection with a POC test kit in accordance with an embodiment of the invention is shown in FIGS. 1-9 and is designated generally by reference numeral 10. Without limiting the scope of the invention, an embodiment is described below with reference to collecting, diluting and dispensing a blood sample. However, it should be appreciated by those of ordinary skill in the art that the apparatus could be used to collect, dilute and dispense any human fluid sample including urine or saliva.

The apparatus 10 resembles a common syringe but has several unique features that enable it to collect a blood sample, mix the blood sample with a buffer solution, and then dispense the diluted blood sample for testing. The apparatus 10 has a distal capillary tube 18 that is constructed to collect by capillary action a precise, predetermined volume of blood from a fingerstick, heelstick, etc. A cavity 12c within the apparatus body 12 is pre-loaded with a precise, predetermined amount of buffer solution “S”. In a closed position, a mixing valve 14 enables admission of blood into the capillary tube 18, but isolates the capillary tube 18 from the internal cavity 12c until the blood sample is ready for dilution. A plunger 20 can be retracted to create and store a vacuum in the internal cavity 12c. In an open position, the mixing valve 14 connects the capillary tube 18 and internal cavity 12c in fluid communication, which enables the vacuum within the internal cavity 12c to draw the blood sample into the internal cavity 12c and mix it with the buffer solution. The diluted blood is then dispensed through the capillary tube 18 by depressing the plunger 20. An expulsion rod 26 increases the dispensing efficiency of the plunger 20 by translating completely through the capillary tube 18. This single, self-contained apparatus achieves all of these functions with minimal skill by the point of care provider.

By way of an exemplary working example, in one preferred embodiment, the glass capillary tube 18 is constructed to accurately collect 27 μL of blood from a fingerstick. The internal cavity 12c of the main body 12 is pre-filled (pre-loaded) and sealed with the precise amount of buffer to create 150 μL of an 18.1% blood/buffer solution. The diluted blood sample is then dispensed onto a lateral flow assay (not shown) to detect the presence of pro-b-type natriuretic peptide (NT-proBNP). The apparatus 10 may be configured with a wide variety and volume of buffer solutions, and the capillary tube 18 may be constructed to collect a wide range of blood volumes, for use in many other POC tests.

In a preferred embodiment, the apparatus 10 has an elongate, cylindrical main body 12 having a longitudinally-extending, central axis “A”, a distal end 12a, proximal end 12b, and internal cavity 12c. A mixing valve 14 is fixed to the distal end 12a of the main body 12. A nosepiece 16 is fixed to the distal side 14a of the valve 14 and includes a central socket. A capillary tube 18 extends from the distal side 16a of the nosepiece 16.

The capillary tube 18 is preferably fixed in the socket of the nosepiece 16 using an adhesive. However, in other embodiments, the nosepiece may be made of an elastomeric material and the diameter of the socket is dimensioned to have an inner diameter slightly smaller than the outer diameter of the capillary tube. In this embodiment, the capillary tube is held in place by a compressive force when the capillary tube is forcibly inserted into the socket.

In the open position, the valve 14 connects the capillary tube 18 and internal cavity 12c in fluid communication. In the closed position, the valve 14 seals the internal cavity 12c, and vents the capillary tube 18 to the atmosphere. Venting enables admission of fluid into the capillary tube 18, which would otherwise be inhibited if the proximal end of the capillary tube 18 was sealed by abutment against a solid surface of the valve gate 34.

The apparatus 10 has a plunger 20, which reciprocates within the main body 12. The plunger 20 arranged along the central axis “A”. The plunger 20 generally comprises an actuator, a seal 24, and an expulsion rod 26. In the preferred embodiment shown in FIGS. 1-9, the actuator comprises an actuator stem 27 and a handle 28 fixed to the proximal end of the stem 27. The seal 24 is seated on the distal end of the stem 27. The seal 24 is preferably made from a compressible, inert elastomer that has an outer diameter slightly larger than the inner diameter of the internal cavity 12c. As described in greater detail below, the plunger 20 reciprocates between a retracted limit position and an extended limit position. As the plunger 20 retracts proximally (backward), it creates a vacuum in the internal cavity 12c if the valve 14 is in the closed position. As the plunger 20 extends distally (forward), it expels the contents of the internal cavity 12c and the capillary tube 18 if the valve is in the open position.

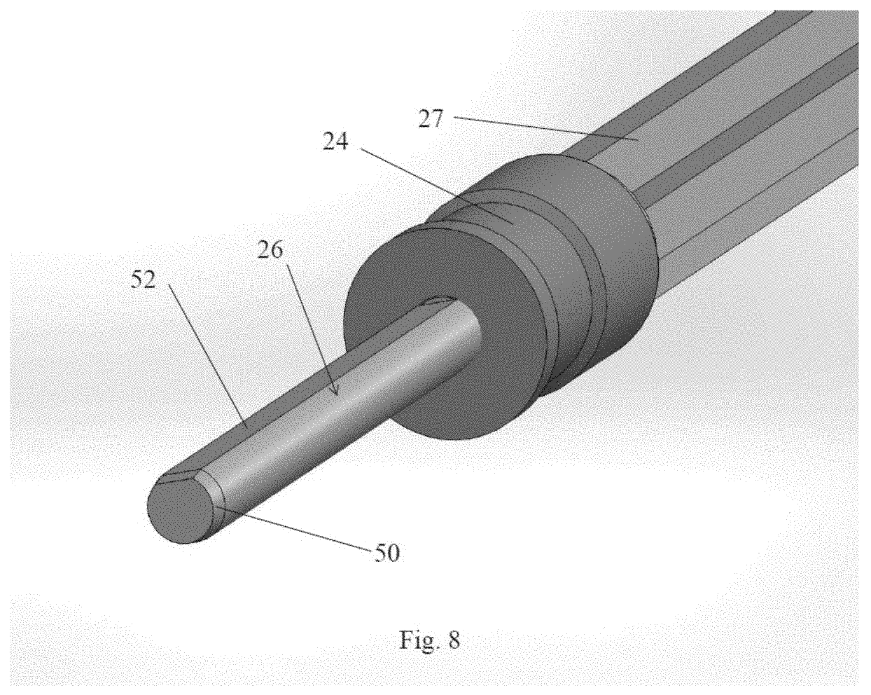

The expulsion rod 26 is preferably integrally formed with the actuator stem 27 and has a diameter much smaller than the inner diameter of the internal cavity 12c, but only slightly smaller than the inner diameter of the capillary tube 18. As best seen in FIGS. 7 and 8, the expulsion rod 26 has a chamfered distal nose 50 and a flat 52 that extends along the length of the expulsion rod 26. As discussed in greater detail below, the flat 52 (and the small clearance between the inner capillary tube wall and expulsion rod 26) enables fluid flow along the outer surface of the expulsion rod during expulsion of the diluted blood sample from the capillary tube 18 even when the expulsion rod is positioned in the mixing valve and/or capillary tube 18. The expulsion rod 26 greatly reduces the volume of the capillary tube during fluid-sample expulsion thereby minimizing the amount of blood sample that remains inside the capillary tube 18.

The expulsion rod 26 may be integrally formed with the actuator stem 27, or may be a separate component connected to the distal end of the actuator stem 27. In either case, the expulsion rod 26 should be co-axial with the central axis “A” so that it can pass freely through the mixing valve in the open position and through the capillary tube 18. The expulsion rod 26 is preferably made from a material, such as Teflon, that will pass smoothly through the transitions between the mixing valve, capillary tube, and cylindrical body.

The mixing valve 14 comprises a valve body 30 having an open-ended, generally-rectangular shape with a distal wall 30a, proximal wall 30b, and lateral side walls 30c, 30d. The inner surfaces of the walls form a rectangular, channel 32 in which a valve gate 34 is movably seated. In the embodiment shown in FIGS. 1-9, the central axis of the rectangular channel 32 is oriented generally-perpendicular to the central axis “A” of the main body 12. A distal bore 36a and a proximal bore 36b extend through the distal wall 30a and proximal wall 30b, respectively, of the valve body 30. The bores 36a, 36b are axially aligned with each other, the expulsion rod 26, the capillary tube 18, and the central axis of the main body 12.

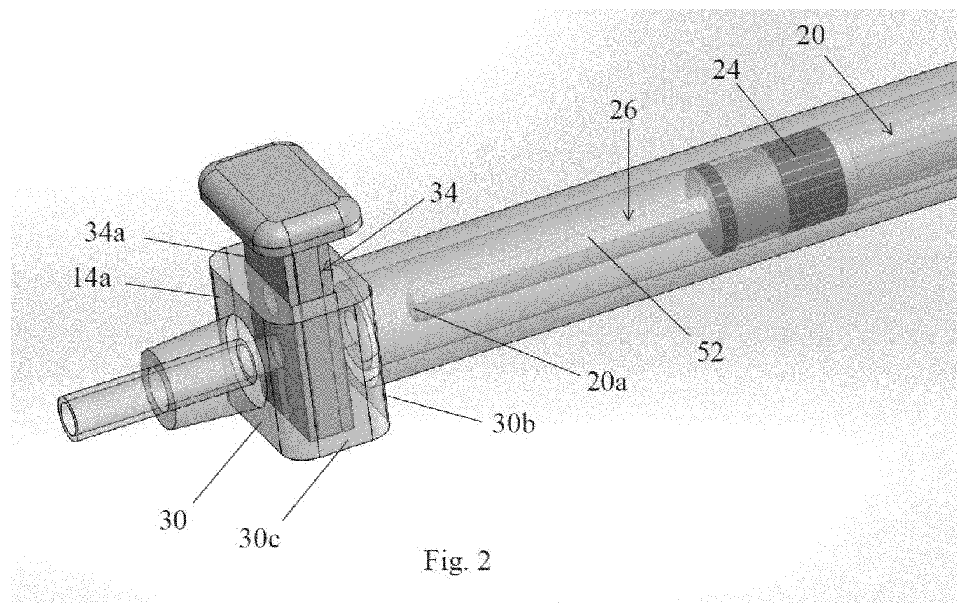

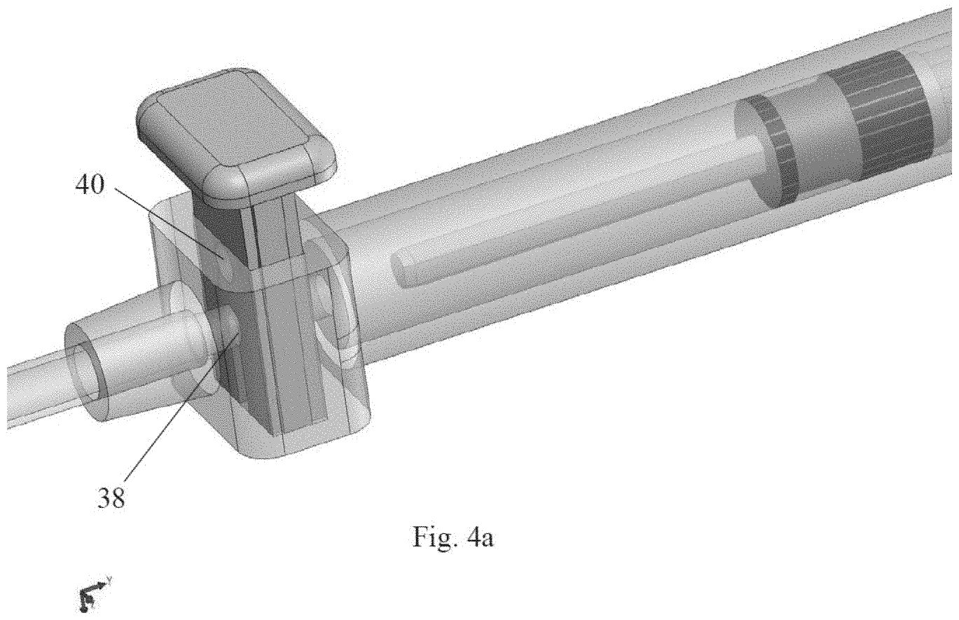

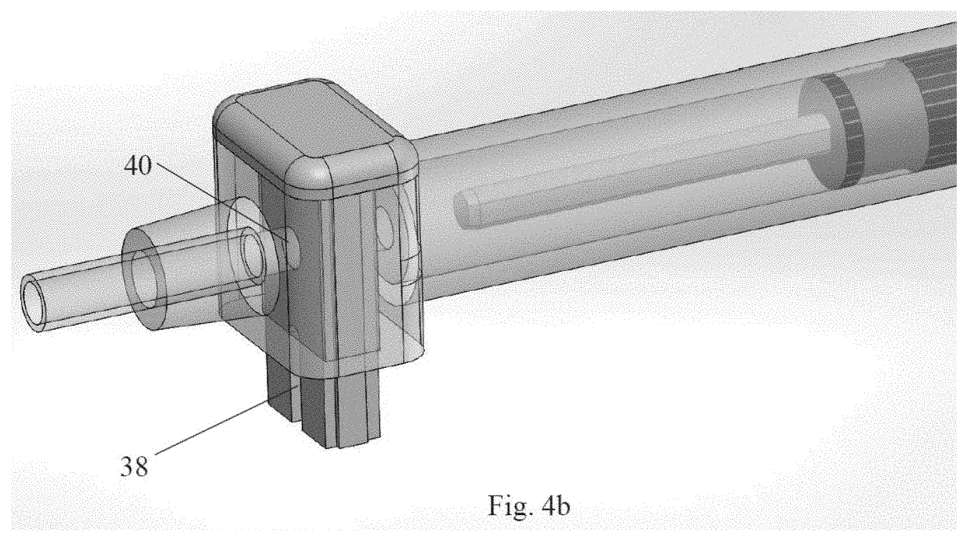

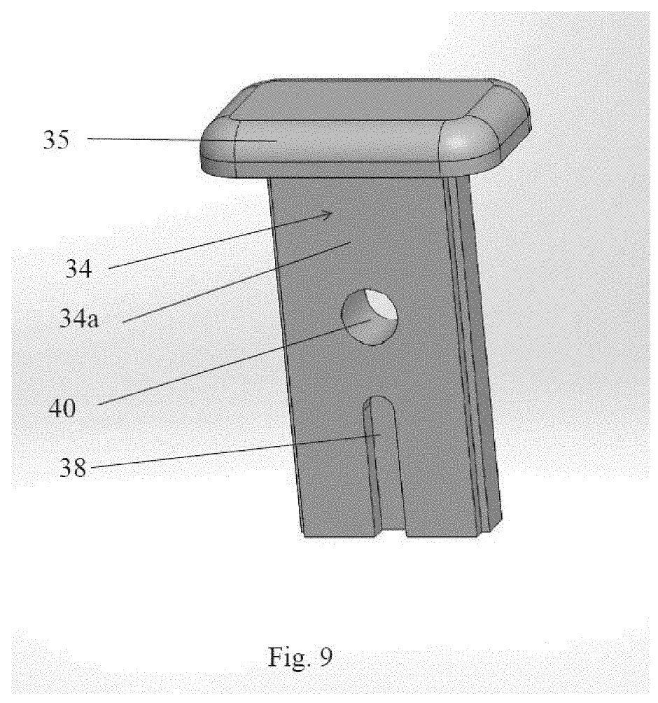

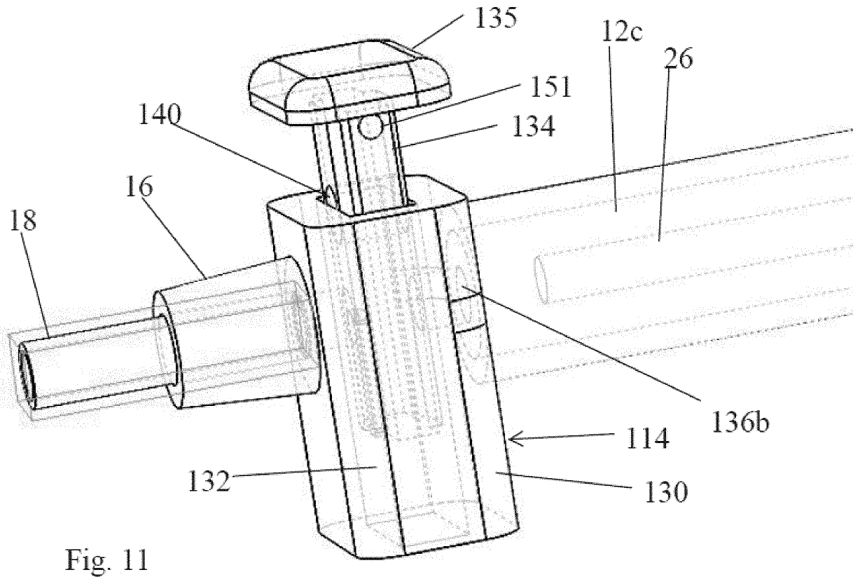

The valve gate 34 has a size and shape that generally compliment the channel 32, so that the gate 34 can freely reciprocate within the channel 32 from a closed position shown in FIGS. 2, 3, 4, 4a, 10 and 11 to an open position shown in FIG. 4b (and still make effective seals with the two sides of the valve body 36a & 36b). The valve gate 34 has a generally-solid construction with an upper button 35, a vent channel 38, and a fluid-flow channel 40. As best seen in FIG. 9, the vent channel 38 extends down the distal face 34a of the valve gate 34 generally along the axis of the rectangular channel 32 of the valve body 30. The vent channel 38 vents the capillary tube 18 and the distal axial bore 36a to the atmosphere when the valve 14 is in the closed position. The fluid-flow channel 40 extends from the distal face 34a to the proximal face 34b of the gate 34. The fluid-flow channel 40 connects the capillary tube 18 in fluid communication with the internal cavity 12c of the main body 12. In a preferred embodiment, the valve gate 34 is made from a polymer material that has a softer durometer than the valve body 30 to ensure that the valve gate 34 makes a good seal with the valve body 30 faces (36a & 36b).

The capillary tube 18 is designed to collect a very small, precise, predetermined amount of fluid, in particular blood, which will then be combined with a predetermined volume of buffer solution “S” contained within the main body internal cavity 12c. For example, in one test, the capillary tube 18 is designed to collect an aliquot of about 27 μL of blood. In the preferred embodiment, the capillary tube 18 is made of glass having an outer diameter slightly smaller than the inner diameter of the nosepiece 16. The capillary tube 18 preferably extends entirely through the nosepiece 16, and abuts the distal face 30a of valve body 30 to form a communication channel with the fluid-flow closed distal bore 36a in the valve body 30.

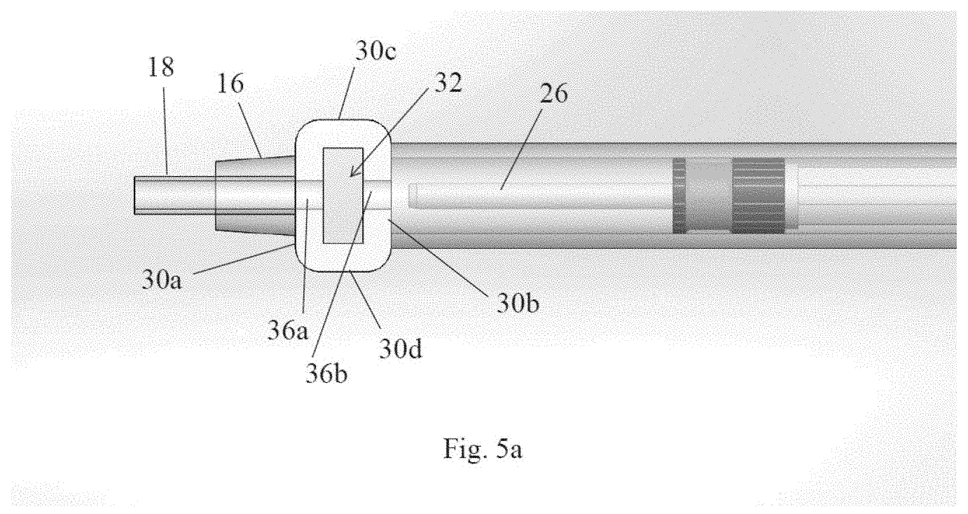

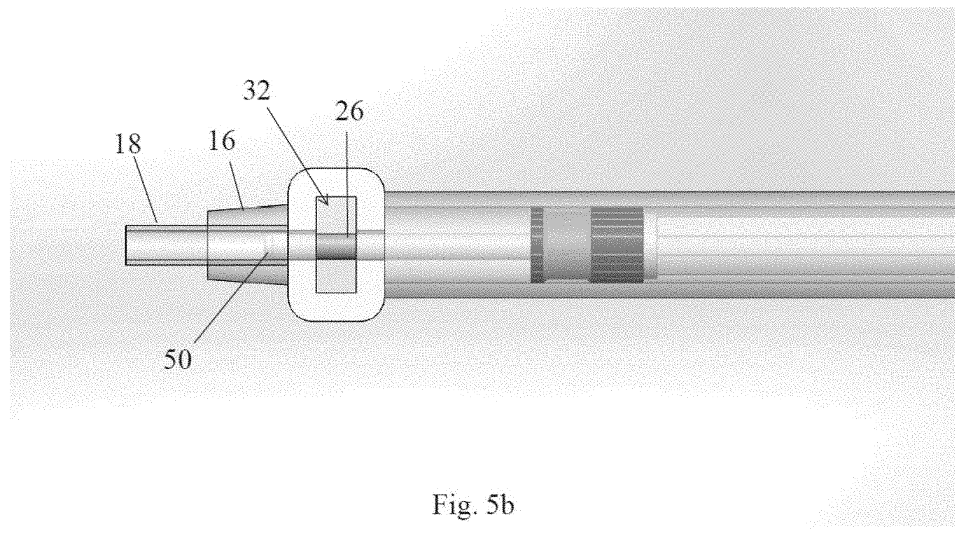

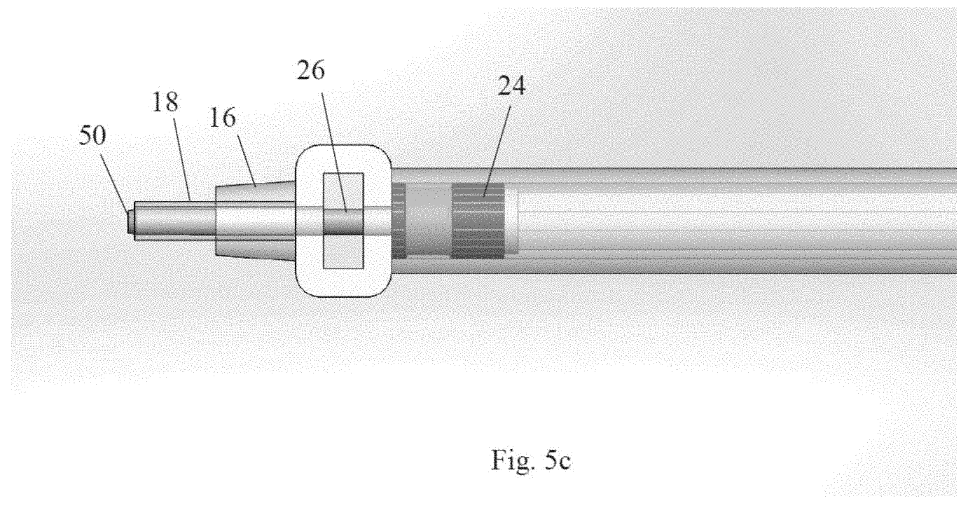

Use of the apparatus to collect, dilute and dispense a blood sample is described with reference to FIGS. 4a-5c. The apparatus 10 is initially provided to the point-of-care provider in the pre-packaged condition shown in FIG. 1, in which a predetermined volume of buffer solution “S” is pre-loaded in the internal cavity 12c of the main body 12. In this condition, the mixing valve is in the closed position better seen in FIG. 4a. When the mixing valve is closed, the internal cavity 12c of the main body 12 is sealed, and the capillary tube 18 is vented to the atmosphere through the vent channel 38.

Next, the provider simply contacts the distal end of the capillary tube 18 to blood from the patient. A precise, predetermined volume of blood is drawn into the capillary tube 18 by capillary action. The provider need only keep the distal tip of the capillary tube immersed in the patient's blood until it automatically stops wicking.

Next, the provider retracts the plunger 20 to create a vacuum in the internal cavity 12c of the main body. To prevent the plunger 20 from rebounding, the stem 27 of the actuator includes a detent 29, which temporarily locks the plunger 20 in the retracted position.

Next, the provider opens the mixing valve by depressing the top button 35 on the valve gate 34 until it abuts the top of the valve body as shown in FIG. 4b. Movement of the valve gate 34 from the closed position to the open position aligns the fluid-flow channel 40 with the distal and proximal axial bores 36a, 36b, which connect the capillary tube 18 in fluid communication with the internal cavity 12c of the main body. The vacuum created by withdrawing the plunger draws the blood sample into the cavity 12c. Release of the differential pressure created by the vacuum causes the blood sample to be jetted into the internal cavity 12c, which mixes the blood sample with the buffer solution “S” to create a diluted blood sample.

Finally, to expel the diluted blood sample, the provider (optionally) inverts the apparatus 10 and depresses the plunger 20 until the seal 24 reaches the distal end of the main body 12. As the plunger 20 expels the diluted blood sample from the main body 12, the expulsion rod 26 passes through the mixing valve 14 and into the capillary tube 18 as best seen in FIG. 5b. The flat 52 on the expulsion rod 26, and the small clearance between the outer surface of the expulsion rod 26 and the inner surface of the capillary tube 18, causes additional mixing and allows the diluted blood sample to flow along the length of the expulsion rod 26 and out of the capillary tube. At the same time, the expulsion rod 26 is pushing diluted blood from the capillary tube 18. Expulsion is complete when the seal bottoms out at the end of the main body 12, and the expulsion rod 26 extends slightly past the distal end of the capillary tube 18, as best seen in FIG. 5c.

The apparatus has particular use with small blood samples requiring precise dilution. Without the expulsion rod 26, a capillary tube would remain filled with a volume of diluted blood sample or a mixture of diluted blood sample & air equal to the volume of the capillary tube 18. In the above-described example, the remaining volume would be 27 μL. Since this volume may be the diluted blood sample or air, the final dispensed volume can vary up to 27 μl. However, because the expulsion rod traverses the entire length of the capillary tube 18, it expels nearly the entire volume of diluted blood sample contained therein. In this example, the remaining volume and the final dispensed volume variability is reduced to about 5 μL, which is due to the flat 52 and expulsion rod/capillary tube interface clearance.

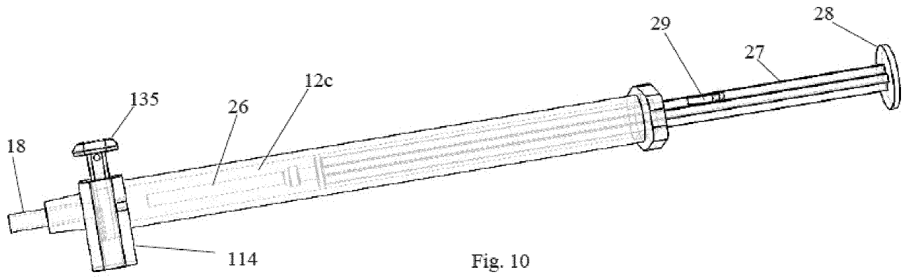

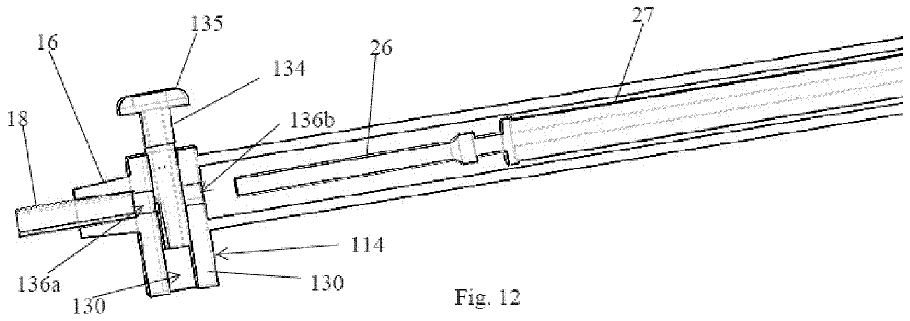

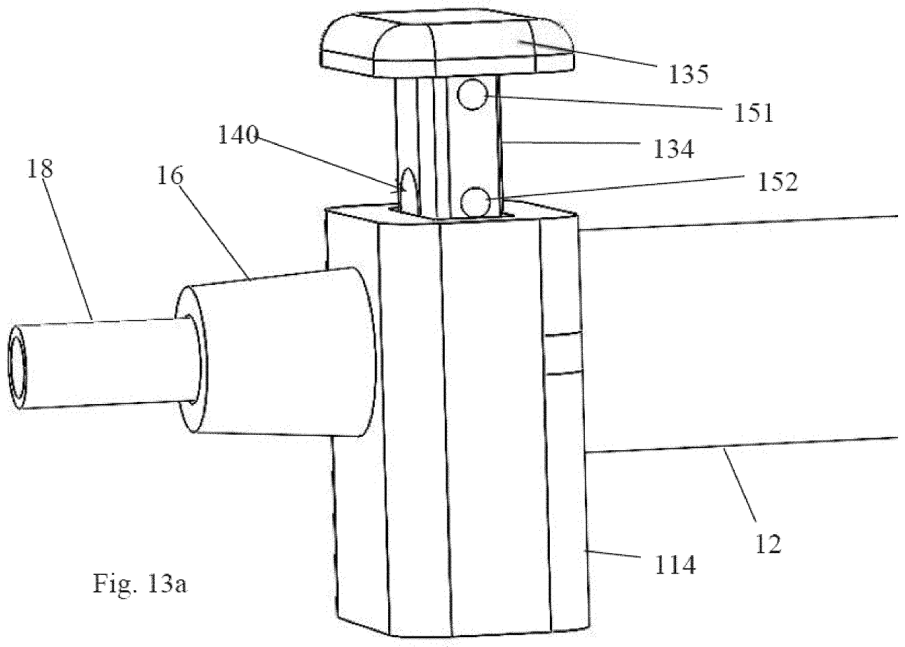

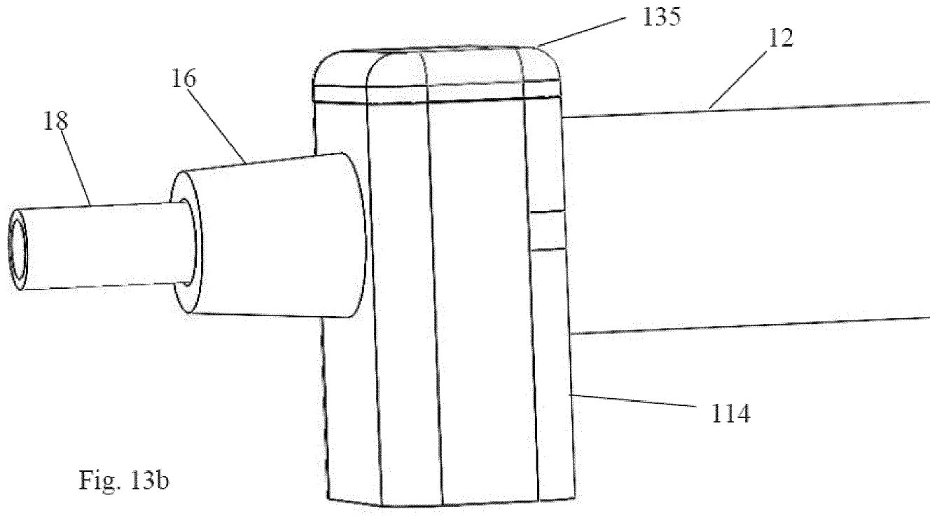

Another preferred embodiment of the apparatus in accordance with the invention is shown in FIGS. 10-13. This embodiment is similar in all respects to the embodiment shown in FIGS. 1-9. However, in this embodiment, the valve includes design features that make it very difficult for the operator to move the valve back to the closed position from the open position. Therefore, this construction helps prevent the operator from using the apparatus a second time.

In this embodiment, the length of the valve body 130 is greater than that shown in FIGS. 1-9 so that the bottom portion of the valve gate 134 does not extend past the bottom of the valve body 130 in the open position. This construction makes it extremely difficult for the operator to push the valve gate 134 back to the closed position by depressing the bottom of the valve gate 134.

In this embodiment, the valve gate 134 also includes a pair of detents 151 and 152. In the closed position, the lower detent 152 engages an interior depression (not shown) on the inside wall of the valve body 130 so as to make it more difficult to move downwardly from that position. Likewise, in the open position, the upper detent 151 engages the interior depression so as to make it more difficult to move upwardly from that position. Preferably, the detent 151 exerts enough locking force to prevent the operator from grasping the sides of the upper button 135 on the valve gate 134 and pulling the valve gate to the closed position. In all other structural and functional aspects, the valve 114 of this embodiment is the same as the valve 14 disclosed in FIGS. 1-9, where similar features are designated with reference numerals including the prefix “1”, while identical features of the other structural elements of the apparatus retain their original reference numeral.

It is to be understood that the description, specific examples and data, while indicating exemplary embodiments, are given by way of illustration and are not intended to limit the present invention. Various changes and modifications within the present invention will become apparent to the skilled artisan from the discussion, disclosure and data contained herein, and thus are considered part of the invention.

1. An apparatus for collecting, diluting and dispensing a patient fluid sample, comprising:
a. an elongate, main body having a longitudinally-extending central axis, a distal end, a proximal end, and an internal cavity containing a diluting solution;
b. a capillary tube at said distal end of said main body;
c. a mixing valve intermediate said capillary tube and said internal cavity for selectively connecting and disconnecting a fluid-flow channel between said capillary tube and said internal cavity; and,
d. a plunger that can reciprocate within said main body between a retracted limit position and a distal limit position for creating negative and positive pressure within said internal cavity.

2. The apparatus recited in claim 1, including a nosepiece on a distal face of said mixing valve, which is constructed and arranged to releasably connect the capillary tube in fluid connection with mixing valve.

3. The apparatus recited in claim 2, wherein said nosepiece is made of an elastomeric material, and has a socket with an inner diameter slightly smaller than the outer diameter of said capillary tube so that a compressive force connects the capillary tube when it is forced into said socket.

4. The apparatus recited in claim 1, said mixing valve having an open position and a closed position, and a vent channel in fluid connection with said capillary tube when said mixing valve is in the closed position.

5. The apparatus recited in claim 1, wherein said mixing valve enables admission of the fluid sample into said capillary tube and isolates said capillary tube from the internal cavity in the closed position, and enables the fluid sample to flow from said capillary tube into said internal cavity in the open position.

6. The apparatus recited in claim 5, said mixing valve including:
a. a valve body having distal, proximal and lateral side walls, a central channel, and a distal and proximal bore that extends axially through the distal and proximal wall, respectively, of the valve body and are positioned coaxially with said central axis;
b. a valve gate having:
i. a size and shape that are complimentary to said central channel of said valve body and slidable within said central channel,
ii. a fluid-flow channel extending axially therethrough that can align with said distal and proximal bores in the valve body when said valve is in the open position;
iii. a vent channel extending down the face of said distal valve body wall that can align with said capillary tube when said valve is in the closed;
c. an actuator button fixed to one end of said valve gate for moving said valve gate from the closed positon to the open position.

7. The apparatus recited in claim 6, wherein said mixing valve is in the open position when said actuator button is depressed to an open limit position abutting said valve body.

8. The apparatus recited in claim 7, wherein said actuator button sits flush with said valve body in the open limit position.

9. The apparatus recited in claim 1, wherein said central cavity of said main body is pre-loaded with a precise, predetermined amount of dilution solution based on the known volume of the capillary tube.

10. The apparatus recited in claim 1, said plunger comprising an actuator stem, a seal seated at the distal end of said stem, and a handle fixed to the proximal end of said stem.

11. The apparatus recited in claim 10, including an expulsion rod fixed to the distal end of said actuator stem, said rod having a diameter slightly smaller than the inner diameter of the capillary tube.

12. The apparatus recited in claim 11, wherein said expulsion rod has a chamfered distal nose and a flat surface that extends along the length of said expulsion rod.

13. The apparatus recited in claim 11, wherein said expulsion rod is arranged co-axially with said central axis so that said expulsion rod extends through the fluid-flow channel in the mixing valve and the capillary tube when said plunger is moved to its distal limit position.

14. The apparatus recited in claim 11, wherein said expulsion rod is made from Teflon and can slide smoothly through the transitions between the main body, mixing valve and capillary tube.

15. An apparatus for collecting, diluting and dispensing a patient fluid sample, comprising:
a. a body having a longitudinally-extending central axis, a distal end, a proximal end, and an internal cavity containing a diluting solution;
b. a capillary tube at said distal end of said body;
c. a valve intermediate said capillary tube and said internal cavity for selectively connecting and disconnecting a fluid-flow connection between said capillary tube and said internal cavity; and,
d. means for transferring the fluid sample in the capillary tube into said internal cavity;
e. first means for mixing the fluid sample and diluting solution within said cavity to form a diluted fluid sample; and
f. means for expelling the diluted fluid sample from said internal cavity and into said capillary tube.

16. The apparatus recited in claim 15, including means for expelling the diluted fluid sample from said capillary tube.

17. The apparatus recited in claim 16, including second means for mixing the diluted fluid sample within said capillary tube as it is being expelled.

18. The apparatus recited in claim 17, including means for venting said capillary tube as the diluted fluid sample is being expelled from said capillary tube.

19. The apparatus recited in claim 15, including means for preventing the apparatus from being re-used by preventing the valve from being closed once it is opened.

20. The apparatus recited in claim 19, wherein said preventing means comprises a detent that retards actuation of the valve and a valve shape that retards manual actuation of the valve.
